SEQUENCE LISTING (231 sequences)

SEQ ID NO: 1 - protein, 113 aa, synthetic construct
  REGION 1..113: note = synthetic sequence
EVQLVESGGGLVQPGGSLRLSCAASGFTFSNYWMNWVRQAPGKGLEWVADIRLKSNNYATHYAESVKGRFTISRDNAKNSLYLQMNSLRAEDTAVYYCVRDGPYWGQGTLVTV

SEQ ID NO: 2 - protein, 125 aa, synthetic construct
  REGION 1..125: note = synthetic sequence
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCVRDGPYWGQGTLVTVSAASTKGPSVFPLA

SEQ ID NO: 3 - protein, 113 aa, synthetic construct
  REGION 1..113: note = synthetic sequence
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCFQGSHVPRTFGGGTKLEIKR

SEQ ID NO: 4 - protein, 113 aa, synthetic construct
  REGION 1..113: note = synthetic sequence
DVVMTQSPLSLPVTLGQPASISCRSSQSIVHSNGNTYLEWFQQRPGQSPRRLIYKISNRFSGVPDRFSGSGSGTDFTLKISRVEAEDVGVYYCFQGSHVPRTFGGGTKLEIKR

SEQ ID NO: 5 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 6 - protein, 120 aa, synthetic construct
  REGION 1..120: note = synthetic sequence
QVQLVQSGAEVKKPGASVKVSCKASGYIFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 7 - protein, 120 aa, synthetic construct
  REGION 1..120: note = synthetic sequence
QVQLVQSGAEVKKPGASVKVSCKASGYNFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 8 - protein, 120 aa, synthetic construct
  REGION 1..120: note = synthetic sequence
QVQLVQSGAEVKKPGASVKVSCKASGYSFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 9 - protein, 120 aa, synthetic construct
  REGION 1..120: note = synthetic sequence
QVQLVQSGAEVKKPGASVKVSCKASGYTFHSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 10 - protein, 120 aa, synthetic construct
  REGION 1..120: note = synthetic sequence
QVQLVQSGAEVKKPGASVKVSCKASGYTFSSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 11 - protein, 120 aa, synthetic construct
  REGION 1..120: note = synthetic sequence
QVQLVQSGAEVKKPGASVKVSCKASGYTFTAYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 12 - protein, 120 aa, synthetic construct
  REGION 1..120: note = synthetic sequence
QVQLVQSGAEVKKPGASVKVSCKASGYTFTGYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 13 - protein, 120 aa, synthetic construct
  REGION 1..120: note = synthetic sequence
QVQLVQSGAEVKKPGASVKVSCKASGYTFTNYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 14 - protein, 120 aa, synthetic construct
  REGION 1..120: note = synthetic sequence
QVQLVQSGAEVKKPGASVKVSCKASGYTFTTYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 15 - protein, 120 aa, synthetic construct
  REGION 1..120: note = synthetic sequence
QVQLVQSGAEVKKPGASVKVSCKASGYTFTSFWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 16 - protein, 120 aa, synthetic construct
  REGION 1..120: note = synthetic sequence
QVQLVQSGAEVKKPGASVKVSCKASGYTFTSHWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 17 - protein, 120 aa, synthetic construct
  REGION 1..120: note = synthetic sequence
QVQLVQSGAEVKKPGASVKVSCKASGYTFTSYWMHWVRQAPGQHLEWMGTIYPRNSDTDYNQKFKARVTMTRDTSTSTVYMELSSLRSEDTAVYYCARPLYYYVTSPPTLLWGQGTLVTV

SEQ ID NO: 18 - protein, 120 aa, synthetic construct
  REGION 1..120: note = synthetic sequence
QVQLVQSGAEVKKPGASVKVSCKASGYTFTSYWMHWVRQAPGQGLEWMGTFYPRNSDTDYNQKFKARVTMTRDTSTSTVYMELSSLRSEDTAVYYCARPLYYYVTSPPTLLWGQGTLVTV

SEQ ID NO: 19 - protein, 120 aa, synthetic construct
  REGION 1..120: note = synthetic sequence
QVQLVQSGAEVKKPGASVKVSCKASGYTFTSYWMHWVRQAPGQGLEWMGTLYPRNSDTDYNQKFKARVTMTRDTSTSTVYMELSSLRSEDTAVYYCARPLYYYVTSPPTLLWGQGTLVTV

SEQ ID NO: 20 - protein, 120 aa, synthetic construct
  REGION 1..120: note = synthetic sequence
QVQLVQSGAEVKKPGASVKVSCKASGYTFTSYWMHWVRQAPGQGLEWMGTNYPRNSDTDYNQKFKARVTMTRDTSTSTVYMELSSLRSEDTAVYYCARPLYYYVTSPPTLLWGQGTLVTV

SEQ ID NO: 21 - protein, 120 aa, synthetic construct
  REGION 1..120: note = synthetic sequence
QVQLVQSGAEVKKPGASVKVSCKASGYTFTSYWMHWVRQAPGQGLEWMGTVYPRNSDTDYNQKFKARVTMTRDTSTSTVYMELSSLRSEDTAVYYCARPLYYYVTSPPTLLWGQGTLVTV

SEQ ID NO: 22 - protein, 120 aa, synthetic construct
  REGION 1..120: note = synthetic sequence
QVQLVQSGAEVKKPGASVKVSCKASGYTFTSYWMHWVRQAPGQGLEWMGTINPRNSDTDYNQKFKARVTMTRDTSTSTVYMELSSLRSEDTAVYYCARPLYYYVTSPPTLLWGQGTLVTV

SEQ ID NO: 23 - protein, 120 aa, synthetic construct
  REGION 1..120: note = synthetic sequence
QVQLVQSGAEVKKPGASVKVSCKASGYTFTSYWMHWVRQAPGQGLEWMGTITPRNSDTDYNQKFKARVTMTRDTSTSTVYMELSSLRSEDTAVYYCARPLYYYVTSPPTLLWGQGTLVTV

SEQ ID NO: 24 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDIDYNQKFKARVTMTRDTSTSTVYMELSSLRSEDTAVYYCARPLYYYVTSPPTLLWGQGTLVTV

SEQ ID NO: 25 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQNFKARVTMTRDTSTSTVYMELSSLRSEDTAVYYCARPLYYYVTSPPTLLWGQGTLVTV

SEQ ID NO: 26 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQRFKARVTMTRDTSTSTVYMELSSLRSEDTAVYYCARPLYYYVTSPPTLLWGQGTLVTV

SEQ ID NO: 27 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTVTRDTSTSTVYMELSSLRSEDTAVYYCARPLYYYVTSPPTLLWGQGTLVTV

SEQ ID NO: 28 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMSRDTSTSTVYMELSSLRSEDTAVYYCARPLYYYVTSPPTLLWGQGTLVTV

SEQ ID NO: 29 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTNTVYMELSSLRSEDTAVYYCARPLYYYVTSPPTLLWGQGTLVTV

SEQ ID NO: 30 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTTTVYMELSSLRSEDTAVYYCARPLYYYVTSPPTLLWGQGTLVTV

SEQ ID NO: 31 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMDLSSLRSEDTAVYYCARPLYYYVTSPPTLLWGQGTLVTV

SEQ ID NO: 32 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMELRSLRSEDTAVYYCARPLYYYVTSPPTLLWGQGTLVTV

SEQ ID NO: 33 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLTSEDTAVYYCARPLYYYVTSPPTLLWGQGTLVTV

SEQ ID NO: 34 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTAYMELSSLRSEDTAVYYCARPLYYYVTSPPTLLWGQGTLVTV

SEQ ID NO: 35 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSDDTAVYYCARPLYYYVTSPPTLLWGQGTLVTV

SEQ ID NO: 36 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSEDTAIYYCARPLYYYVTSPPTLLWGQGTLVTV

SEQ ID NO: 37 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSIVYMELSSLRSEDTAVYYCARPLYYYVTSPPTLLWGQGTLVTV

SEQ ID NO: 38 - protein, 120 aa, synthetic construct
  REGION 1..120: note = synthetic sequence
QVQLVQSGAEVKKPGASVKVSCKASGYTFTSYFMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 39 - protein, 120 aa, synthetic construct
  REGION 1..120: note = synthetic sequence
QVQLVQSGAEVKKPGASVKVSCKASGYTFTSYWMHWVRQAPGQGLEWMGTIDPRNSDTDYNQKFKARVTMTRDTSTSTVYMELSSLRSEDTAVYYCARPLYYYVTSPPTLLWGQGTLVTV

SEQ ID NO: 40 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTRTVYMELSSLRSEDTAVYYCARPLYYYVTSPPTLLWGQGTLVTV

SEQ ID NO: 41 - protein, 120 aa, synthetic construct
  REGION 1..120: note = synthetic sequence
QVQLVESGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 42 - protein, 120 aa, synthetic construct
  REGION 1..120: note = synthetic sequence
QVQLVQSGAEVKKPGASVKVSCKASGYTFTDYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 43 - protein, 120 aa, synthetic construct
  REGION 1..120: note = synthetic sequence
QVQLVQSGAEVKKPGASVKVSCKASGYTFTSYWIHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 44 - protein, 120 aa, synthetic construct
  REGION 1..120: note = synthetic sequence
QVQLVQSGAEVKKPGASVKVSCKASGYTFTSYWLHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 45 - protein, 120 aa, synthetic construct
  REGION 1..120: note = synthetic sequence
QVQLVQSGAEVKKPGASVKVSCKASGYTFTSYWMNWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 46 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDFNQKFKARVTMTRDTSTSTVYMELSSLRSEDTAVYYCARPLYYYVTSPPTLLWGQGTLVTV

SEQ ID NO: 47 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDHNQKFKARVTMTRDTSTSTVYMELSSLRSEDTAVYYCARPLYYYVTSPPTLLWGQGTLVTV

SEQ ID NO: 48 - protein, 120 aa, synthetic construct
  REGION 1..120: note = synthetic sequence
QVQLVQSGAEVKKPGASVKVSCKASGYTFTSYWMHWVRQAPGQGLEWMGTIYPRNSDTDYNQKFKARVTMTRDTSTSTTYMELSSLRSEDTAVYYCARPLYYYVTSPPTLLWGQGTLVTV

SEQ ID NO: 49 - protein, 120 aa, synthetic construct
  REGION 1..120: note = synthetic sequence
QVQLVQSGAEVKKPGASVKVSCKASGYTFTSHWMHWVRQAPGQGLEWMGTIYPRNSDTDYNQKFKARVTMTRDTSTSIVYMELSSLRSEDTAVYYCARPLYYYVTSPPTLLWGQGTLVTV

SEQ ID NO: 50 - protein, 122 aa, synthetic construct
  REGION 1..122: note = synthetic sequence
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 51 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQCNSYPFTFGSGTKLEIKR

SEQ ID NO: 52 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQVNSYPFTFGSGTKLEIKR

SEQ ID NO: 53 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQINSYPFTFGSGTKLEIKR

SEQ ID NO: 54 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQLNSYPFTFGSGTKLEIKR

SEQ ID NO: 55 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQANSYPFTFGSGTKLEIKR

SEQ ID NO: 56 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQSNSYPFTFGSGTKLEIKR

SEQ ID NO: 57 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQASTYPFTFGSGTKLEIKR

SEQ ID NO: 58 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQASAYPFTFGSGTKLEIKR

SEQ ID NO: 59 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQARTYPFTFGSGTKLEIKR

SEQ ID NO: 60 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQARAYPFTFGSGTKLEIKR

SEQ ID NO: 61 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 62 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAAAYPFTFGSGTKLEIKR

SEQ ID NO: 63 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAQTYPFTFGSGTKLEIKR

SEQ ID NO: 64 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAQAYPFTFGSGTKLEIKR

SEQ ID NO: 65 - protein, 108 aa, synthetic construct
  REGION 1..108: note = synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAYSYPFTFGSGTKLEIKR

SEQ ID NO: 66 - protein, 108 aa, synthetic construct
  REGION 1..108: note = synthetic sequence
DIQLTQSPSFLSASVGDRVTITCRASQGISSYLAWYQQKPGKAPKLLIYAASTLQSGVPSRFSGSGSGTEFTLTISSLQPEDFATYYCQQCNSYPFTFGSGTKLEIKR

SEQ ID NO: 67 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 68 - protein, 139 aa, synthetic construct; identical to SEQ ID NO: 67
  REGION 1..139: note = synthetic sequence

SEQ ID NO: 69 - protein, 139 aa, synthetic construct
  REGION 1..139: note = synthetic sequence
MRAWIFFLLCLAGRALAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 70 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTAYMELSSLRSEDTAVYYCARPLYYYVTSPPTLLWGQGTLVTVSS

SEQ ID NO: 71 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIDPRNSDTDYNQKFKARVTMTRDTSTSTVYMELSSLRSEDTAVYYCARPLYYYVTSPPTLLWGQGTLVTVSS

SEQ ID NO: 72 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTITPRNSDTDYNQKFKARVTMTRDTSTSTVYMELSSLRSEDTAVYYCARPLYYYVTSPPTLLWGQGTLVTVSS

SEQ ID NO: 73 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTINPRNSDTDYNQKFKARVTMTRDTSTSTVYMELSSLRSEDTAVYYCARPLYYYVTSPPTLLWGQGTLVTVSS

SEQ ID NO: 74 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMDLSSLRSEDTAVYYCARPLYYYVTSPPTLLWGQGTLVTVSS

SEQ ID NO: 75 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSDDTAVYYCARPLYYYVTSPPTLLWGQGTLVTVSS

SEQ ID NO: 76 - protein, 139 aa, synthetic construct
  REGION 1..139: note = synthetic sequence
MRAWIFFLLCLAGRALAQVQLVQSGAEVKKPGASVKVSCKASGYTFTSYFMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 77 - protein, 139 aa, synthetic construct
  REGION 1..139: note = synthetic sequence
MRAWIFFLLCLAGRALAQVQLVQSGAEVKKPGASVKVSCKASGYTFTGYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 78 - protein, 139 aa, synthetic construct
  REGION 1..139: note = synthetic sequence
MRAWIFFLLCLAGRALAQVQLVQSGAEVKKPGASVKVSCKASGYTFTAYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 79 - protein, 139 aa, synthetic construct
  REGION 1..139: note = synthetic sequence
MRAWIFFLLCLAGRALAQVQLVQSGAEVKKPGASVKVSCKASGYTFTNYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 80 - protein, 139 aa, synthetic construct
  REGION 1..139: note = synthetic sequence
MRAWIFFLLCLAGRALAQVQLVQSGAEVKKPGASVKVSCKASGYTFTTYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 81 - protein, 139 aa, synthetic construct
  REGION 1..139: note = synthetic sequence
MRAWIFFLLCLAGRALAQVQLVQSGAEVKKPGASVKVSCKASGYTFTSHWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 82 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQHLEWMGTIYPRNSDTDYNQKFKARVTMTRDTSTSTVYMELSSLRSEDTAVYYCARPLYYYVTSPPTLLWGQGTLVTVSS

SEQ ID NO: 83 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAIYYCARPLYYYVTSPPTLLWGQGTLVTVSS

SEQ ID NO: 84 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTNTVYMELSSLRSEDTAVYYCARPLYYYVTSPPTLLWGQGTLVTVSS

SEQ ID NO: 85 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTTTVYMELSSLRSEDTAVYYCARPLYYYVTSPPTLLWGQGTLVTVSS

SEQ ID NO: 86 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTRTVYMELSSLRSEDTAVYYCARPLYYYVTSPPTLLWGQGTLVTVSS

SEQ ID NO: 87 - protein, 139 aa, synthetic construct
  REGION 1..139: note = synthetic sequence
MRAWIFFLLCLAGRALAQVQLVQSGAEVKKPGASVKVSCKASGYTFSSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 88 - protein, 139 aa, synthetic construct
  REGION 1..139: note = synthetic sequence
MRAWIFFLLCLAGRALAQVQLVQSGAEVKKPGASVKVSCKASGYTFHSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 89 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMSRDTSTSTVYMELSSLRSEDTAVYYCARPLYYYVTSPPTLLWGQGTLVTVSS

SEQ ID NO: 90 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLTSEDTAVYYCARPLYYYVTSPPTLLWGQGTLVTVSS

SEQ ID NO: 91 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTVTRDTSTSTVYMELSSLRSEDTAVYYCARPLYYYVTSPPTLLWGQGTLVTVSS

SEQ ID NO: 92 - protein, 139 aa, synthetic construct
  REGION 1..139: note = synthetic sequence
MRAWIFFLLCLAGRALAQVQLVQSGAEVKKPGASVKVSCKASGYNFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 93 - protein, 139 aa, synthetic construct
  REGION 1..139: note = synthetic sequence
MRAWIFFLLCLAGRALAQVQLVQSGAEVKKPGASVKVSCKASGYIFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 94 - protein, 139 aa, synthetic construct
  REGION 1..139: note = synthetic sequence
MRAWIFFLLCLAGRALAQVQLVQSGAEVKKPGASVKVSCKASGYSFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 95 - protein, 139 aa, synthetic construct
  REGION 1..139: note = synthetic sequence
MRAWIFFLLCLAGRALAQVQLVQSGAEVKKPGASVKVSCKASGYTFTSFWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 96 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTNYPRNSDTDYNQKFKARVTMTRDTSTSTVYMELSSLRSEDTAVYYCARPLYYYVTSPPTLLWGQGTLVTVSS

SEQ ID NO: 97 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTLYPRNSDTDYNQKFKARVTMTRDTSTSTVYMELSSLRSEDTAVYYCARPLYYYVTSPPTLLWGQGTLVTVSS

SEQ ID NO: 98 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTVYPRNSDTDYNQKFKARVTMTRDTSTSTVYMELSSLRSEDTAVYYCARPLYYYVTSPPTLLWGQGTLVTVSS

SEQ ID NO: 99 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTFYPRNSDTDYNQKFKARVTMTRDTSTSTVYMELSSLRSEDTAVYYCARPLYYYVTSPPTLLWGQGTLVTVSS

SEQ ID NO: 100 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDIDYNQKFKARVTMTRDTSTSTVYMELSSLRSEDTAVYYCARPLYYYVTSPPTLLWGQGTLVTVSS

SEQ ID NO: 101 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQRFKARVTMTRDTSTSTVYMELSSLRSEDTAVYYCARPLYYYVTSPPTLLWGQGTLVTVSS

SEQ ID NO: 102 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQNFKARVTMTRDTSTSTVYMELSSLRSEDTAVYYCARPLYYYVTSPPTLLWGQGTLVTVSS

SEQ ID NO: 103 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELRSLRSEDTAVYYCARPLYYYVTSPPTLLWGQGTLVTVSS

SEQ ID NO: 104 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTTYMELSSLRSEDTAVYYCARPLYYYVTSPPTLLWGQGTLVTVSS

SEQ ID NO: 105 - protein, 139 aa, synthetic construct
  REGION 1..139: note = synthetic sequence
MRAWIFFLLCLAGRALAQVQLVESGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 106 - protein, 139 aa, synthetic construct
  REGION 1..139: note = synthetic sequence
MRAWIFFLLCLAGRALAQVQLVQSGAEVKKPGASVKVSCKASGYTFTDYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 107 - protein, 139 aa, synthetic construct
  REGION 1..139: note = synthetic sequence
MRAWIFFLLCLAGRALAQVQLVQSGAEVKKPGASVKVSCKASGYTFTSYWIHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 108 - protein, 139 aa, synthetic construct
  REGION 1..139: note = synthetic sequence
MRAWIFFLLCLAGRALAQVQLVQSGAEVKKPGASVKVSCKASGYTFTSYWLHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 109 - protein, 139 aa, synthetic construct
  REGION 1..139: note = synthetic sequence
MRAWIFFLLCLAGRALAQVQLVQSGAEVKKPGASVKVSCKASGYTFTSYWMNWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 110 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDHNQKFKARVTMTRDTSTSTVYMELSSLRSEDTAVYYCARPLYYYVTSPPTLLWGQGTLVTVSS

SEQ ID NO: 111 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDFNQKFKARVTMTRDTSTSTVYMELSSLRSEDTAVYYCARPLYYYVTSPPTLLWGQGTLVTVSS

SEQ ID NO: 112 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSIVYMELSSLRSEDTAVYYCARPLYYYVTSPPTLLWGQGTLVTVSS

SEQ ID NO: 113 - protein, 139 aa, synthetic construct
  REGION 1..139: note = synthetic sequence
MRAWIFFLLCLAGRALAQVQLVQSGAEVKKPGASVKVSCKASGYTFTSHWMHWVRQAPGQGLEWMGTIYPRNSDTDYNQKFKARVTMTRDTSTSIVYMELSSLRSEDTAVYYCARPLYYYVTSPPTLLWGQGTLVTVSS

SEQ ID NO: 114 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYITSPPTLLWGQGTLVTVSS

SEQ ID NO: 115 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYLTSPPTLLWGQGTLVTVSS

SEQ ID NO: 116 - protein, 139 aa, synthetic construct; identical to SEQ ID NO: 115
  REGION 1..139: note = synthetic sequence

SEQ ID NO: 117 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYDTSPPTLLWGQGTLVTVSS

SEQ ID NO: 118 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYFTSPPTLLWGQGTLVTVSS

SEQ ID NO: 119 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYATSPPTLLWGQGTLVTVSS

SEQ ID NO: 120 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYGTSPPTLLWGQGTLVTVSS

SEQ ID NO: 121 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLFWGQGTLVTVSS

SEQ ID NO: 122 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLVWGQGTLVTVSS

SEQ ID NO: 123 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLPWGQGTLVTVSS

SEQ ID NO: 124 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYLTSPPTLFWGQGTLVTVSS

SEQ ID NO: 125 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYVTSPPTLLWGQGTLVTVSS

SEQ ID NO: 126 - protein, 139 aa, synthetic construct
  REGION 1..139: note = synthetic sequence
MRAWIFFLLCLAGRALAQVQLMQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 127 - protein, 139 aa, synthetic construct; identical to SEQ ID NO: 121
  REGION 1..139: note = synthetic sequence

SEQ ID NO: 128 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGIIYPRNSDTDYNQKFKARVTMTRDTSTSTVYMELSSLRSEDTAVYYCARPLYYYVTSPPTLLWGQGTLVTVSS

SEQ ID NO: 129 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVISPPTLLWGQGTLVTVSS

SEQ ID NO: 130 - protein, 139 aa, synthetic construct; identical to SEQ ID NO: 67
  REGION 1..139: note = synthetic sequence

SEQ ID NO: 131 - protein, 139 aa, synthetic construct
  REGION 1..139: note = synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 132 - protein, 139 aa, synthetic construct
  REGION 1..139: note = synthetic sequence
MRAWIFFLLCLAGRALAQVQLVQSGAEVKKPGASVKVSCKASGYTFTSYWMHWMRQAPGQGLEWMGTIYPRNSDTDYNQKFKARVTMTRDTSTSTVYMELSSLRSEDTAVYYCARPLYYYVTSPPTLFWGQGTLVTVSS

SEQ ID NO: 133 - protein, 139 aa, synthetic construct
  REGION 1..139: note = synthetic sequence
MRAWIFFLLCLAGRALAQVQLVQSGAEVKKPGASVKVSCKASGYTFTSYWMHWMRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 134 - protein, 139 aa, synthetic construct
  REGION 1..139: note = synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLLWGQGTLVTVSS

SEQ ID NO: 135 - protein, 139 aa, synthetic construct
  REGION 1..139: note = synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYVTSPPTLFWGQGTLVTVSS

SEQ ID NO: 136 - protein, 139 aa, synthetic construct; identical to SEQ ID NO: 131
  REGION 1..139: note = synthetic sequence

SEQ ID NO: 137 - protein, 139 aa, synthetic construct
  REGION 1..139: note = synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 138 - protein, 139 aa, synthetic construct
  REGION 1..139: note = synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYLTSPPTLLWGQGTLVTVSS

SEQ ID NO: 139 - protein, 139 aa, synthetic construct
  REGION 1..139: note = synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYVTSPPTLFWGQGTLVTVSS

SEQ ID NO: 140 - protein, 139 aa, synthetic construct; identical to SEQ ID NO: 137
  REGION 1..139: note = synthetic sequence

SEQ ID NO: 141 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQCNSYPFTFGSGTKLEIKR

SEQ ID NO: 142 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQSYPFTFGSGTKLEIKR

SEQ ID NO: 143 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQANSYPFTFGSGTKLEIKR

SEQ ID NO: 144 - protein, 125 aa, synthetic construct; identical to SEQ ID NO: 143
  REGION 1..125: note = synthetic sequence

SEQ ID NO: 145 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQVNSYPFTFGSGTKLEIKR

SEQ ID NO: 146 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQINSYPFTFGSGTKLEIKR

SEQ ID NO: 147 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQLNSYPFTFGSGTKLEIKR

SEQ ID NO: 148 - protein, 125 aa, synthetic construct; identical to SEQ ID NO: 143
  REGION 1..125: note = synthetic sequence

SEQ ID NO: 149 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQSNSYPFTFGSGTKLEIKR

SEQ ID NO: 150 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQASTYPFTFGSGTKLEIKR

SEQ ID NO: 151 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQASAYPFTFGSGTKLEIKR

SEQ ID NO: 152 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQARTYPFTFGSGTKLEIKR

SEQ ID NO: 153 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQARAYPFTFGSGTKLEIKR

SEQ ID NO: 154 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 155 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKAYPFTFGSGTKLEIKR

SEQ ID NO: 156 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQTYPFTFGSGTKLEIKR

SEQ ID NO: 157 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQAYPFTFGSGTKLEIKR

SEQ ID NO: 158 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQYSSYPFTFGSGTKLEIKR

SEQ ID NO: 159 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQANTYPFTFGSGTKLEIKR

SEQ ID NO: 160 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWAFTRHTGVPSRFSGSGSGTEFTLTISSLQPEDFATYYCQQAQSYPFTFGSGTKLEIKR

SEQ ID NO: 161 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWAATRHTGVPSRFSGSGSGTEFTLTISSLQPEDFATYYCQQAQSYPFTFGSGTKLEIKR

SEQ ID NO: 162 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWAPTRHTGVPSRFSGSGSGTEFTLTISSLQPEDFATYYCQQAQSYPFTFGSGTKLEIKR

SEQ ID NO: 163 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWATTRHTGVPSRFSGSGSGTEFTLTISSLQPEDFATYYCQQAQSYPFTFGSGTKLEIKR

SEQ ID NO: 164 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWAHTRHTGVPSRFSGSGSGTEFTLTISSLQPEDFATYYCQQAQSYPFTFGSGTKLEIKR

SEQ ID NO: 165 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWANTRHTGVPSRFSGSGSGTEFTLTISSLQPEDFATYYCQQAQSYPFTFGSGTKLEIKR

SEQ ID NO: 166 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYWAYTRHTGVPSRFSGSGSGTEFTLTISSLQPEDFATYYCQQAQSYPFTFGSGTKLEIKR

SEQ ID NO: 167 - protein, 125 aa, synthetic construct
  REGION 1..125: note = synthetic sequence
MRAWIFFLLCLAGRALADIQLTQSPSFLSASVGDRVTITCKASQDVRTAVAWYQQKPGKAPKLLIYWASTRHTGVPSRFSGSGSGTEFTLTISSLQPEDFATYYCQQAQSYPFTFGSGTKLEIKR

SEQ ID NO: 168 - protein, 125 aa, synthetic construct
  REGION 1..125: note = synthetic sequence
MRAWIFFLLCLAGRALADIQLTQSPSFLSASVGDRVTITCKASQDVGTAVAWYQQKPGKAPKLLIYVASTRHTGVPSRFSGSGSGTEFTLTISSLQPEDFATYYCQQAQSYPFTFGSGTKLEIKR

SEQ ID NO: 169 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQCNSYPFTFGSGTKLEIKR

SEQ ID NO: 170 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQCNNYPFTFGSGTKLEIKR

SEQ ID NO: 171 - protein, 125 aa, synthetic construct; identical to SEQ ID NO: 154
  REGION 1..125: note = synthetic sequence

SEQ ID NO: 172 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQANNYPFTFGSGTKLEIKR

SEQ ID NO: 173 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQAQSYPFTFGSGTKLEIKR

SEQ ID NO: 174 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVAFTLQSGVPSRFSGSGSGTEFTLTISSLQPEDFATYYCQQCNSYPFTFGSGTKLEIKR

SEQ ID NO: 175 - protein, 125 aa, synthetic construct
  REGION 1..125: note = synthetic sequence
MRAWIFFLLCLAGRALADIQLTQSPSFLSASVGDRVTITCRASQGIRSYLAWYQQKPGKAPKLLIYVAFTLQSGVPSRFSGSGSGTEFTLTISSLQPEDFATYYCQQCNNYPFTFGSGTKLEIKR

SEQ ID NO: 176 - protein, 139 aa, synthetic construct
  REGION 1..139: note = synthetic sequence
MRAWIFFLLCLAGRALAQVQLVQSGAEVKKPGASVKVSCKASGYTFTSYYMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 177 - protein, 139 aa, synthetic construct
  REGION 1..139: note = synthetic sequence
MRAWIFFLLCLAGRALAQVQLVQSGAEVKKPGASVKVSCKASGYTFTSYWMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 178 - protein, 144 aa, synthetic construct
  REGION 1..144: note = synthetic sequence
MRAWIFFLLCLAGRALAEVQLVESGGGLVQPGGSLRLSCAASGFTFSNYWMNWVRQAPGKGLEWVADIRLKSNNYATHYAESVKGRFTISRDNAKNSLYLQMNSLRAEDTAVYYCVRDGPYWGQGTLVTVSAASTKGPSVFPLA

SEQ ID NO: 179 - protein, 144 aa, synthetic construct
  REGION 1..144: note = synthetic sequence
MRAWIFFLLCLAGRALAEVQLVESGGGLVQPGGSLRLSCAASGFTFSNYWMNWVRQAPEKGLEWVADIRLKSNNYATHYAESVKGRFTISRDDAKSSLYLQMNNLRAEDTAVYYCVRDGPYWGQGTLVTVSAASTKGPSVFPLA

SEQ ID NO: 180 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDGPYWGQGTLVTVSAASTKGPSVFPLA

SEQ ID NO: 181 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKSSVYLQMNSLRAEDTAVYYCVRDGPYWGQGTLVTVSAASTKGPSVFPLA

SEQ ID NO: 182 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDAPYWGQGTLVTVSAASTKGPSVFPLA

SEQ ID NO: 183 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDSPYWGQGTLVTVSAASTKGPSVFPLA

SEQ ID NO: 184 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRGGPYWGQGTLVTVSAASTKGPSVFPLA

SEQ ID NO: 185 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRSGPYWGQGTLVTVSAASTKGPSVFPLA

SEQ ID NO: 186 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRTGPYWGQGTLVTVSAASTKGPSVFPLA

SEQ ID NO: 187 - protein, 144 aa, synthetic construct
  REGION 1..144: note = synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRMGPYWGQGTLVTVSAASTKGPSVFPLA

SEQ ID NO: 188 - protein, 125 aa, synthetic construct; identical to SEQ ID NO: 2
  REGION 1..125: note = synthetic sequence

SEQ ID NO: 189 - protein, 113 aa, synthetic construct; identical to SEQ ID NO: 4
  REGION 1..113: note = synthetic sequence

SEQ ID NO: 190 - protein, 130 aa, synthetic construct
  REGION 1..130: note = synthetic sequence
MRAWIFFLLCLAGRALADVVMTQSPLSLPVTLGQPASISCRSSQSLVYSDGNTYLNWFQQRPGQSPRRLIYKVSNRDSGVPDRFSGSGSGTDFTLKISRVEAEDVGVYYCFQGSHVPRTFGGGTKLEIKR

SEQ ID NO: 191 - protein, 130 aa, synthetic construct
  REGION 1..130: note = synthetic sequence
MRAWIFFLLCLAGRALADVVMTQSPLSLPVTLGQPASISCRSSQSIVHSNGNTYLEWYLQRPGQSPRLLIYKISNRFSGVPDRFSGSGSGTDFTLKISRVEAEDVGVYYCFQGSHVPRTFGGGTKLEIKR

SEQ ID NO: 192 - protein, 130 aa, synthetic construct
  REGION 1..130: note = synthetic sequence
MRAWIFFLLCLAGRALADVLMTQSPLSLPVTLGQPASISCRSSQSIVHSNGNTYLEWYQQRPGQSPRLLIYKISNRFSGVPDRFSGSGSGTDFTLKISRVEAEDVGVYYCFQGSHVPRTFGGGTKLEIKR

SEQ ID NO: 193 - protein, 130 aa, synthetic construct
  REGION 1..130: note = synthetic sequence
MRAWIFFLLCLAGRALADVVMTQSPLSLPVTLGQPASISCRSSQSIVHSNGNTYLEWYQQRPGQSPRLLIYKISNRFSGVPDRFSGSGSGTDFTLKISRVEAEDVGVYYCFQGSHVPRTFGGGTKLEIKR

SEQ ID NO: 194 - protein, 130 aa, synthetic construct
  REGION 1..130: note = synthetic sequence
MRAWIFFLLCLAGRALADVVMTQSPLSLPVTLGQPASISCRSSQSIVHSQGNTYLEWYQQRPGQSPRLLIYKISNRFSGVPDRFSGSGSGTDFTLKISRVEAEDVGVYYCFQGSHVPRTFGGGTKLEIKR

SEQ ID NO: 195 - protein, 130 aa, synthetic construct
  REGION 1..130: note = synthetic sequence
MRAWIFFLLCLAGRALADVVMTQSPLSLPVTLGQPASISCRSSQSIVHSLGNTYLEWYQQRPGQSPRLLIYKISNRFSGVPDRFSGSGSGTDFTLKISRVEAEDVGVYYCFQGSHVPRTFGGGTKLEIKR

SEQ ID NO: 196 - protein, 130 aa, synthetic construct
  REGION 1..130: note = synthetic sequence
MRAWIFFLLCLAGRALADVVMTQSPLSLPVTLGQPASISCRSSQSIVHSAGNTYLEWYQQRPGQSPRLLIYKISNRFSGVPDRFSGSGSGTDFTLKISRVEAEDVGVYYCFQGSHVPRTFGGGTKLEIKR

SEQ ID NO: 197 - protein, 130 aa, synthetic construct
  REGION 1..130: note = synthetic sequence
MRAWIFFLLCLAGRALADVVMTQSPLSLPVTLGQPASISCRSSQSIVHSSGNTYLEWYQQRPGQSPRLLIYKISNRFSGVPDRFSGSGSGTDFTLKISRVEAEDVGVYYCFQGSHVPRTFGGGTKLEIKR

SEQ ID NO: 198 - protein, 130 aa, synthetic construct
  REGION 1..130: note = synthetic sequence
MRAWIFFLLCLAGRALADVVMTQSPLSLPVTLGQPASISCRSSQSIVHSEGNTYLEWYQQRPGQSPRLLIYKISNRFSGVPDRFSGSGSGTDFTLKISRVEAEDVGVYYCFQGSHVPRTFGGGTKLEIKR

SEQ ID NO: 199 - protein, 130 aa, synthetic construct
  REGION 1..130: note = synthetic sequence
MRAWIFFLLCLAGRALADVVMTQSPLSLPVTLGQPASISCRSSQSIVHSTGNTYLEWYQQRPGQSPRLLIYKISNRFSGVPDRFSGSGSGTDFTLKISRVEAEDVGVYYCFQGSHVPRTFGGGTKLEIKR

SEQ ID NO: 200 - protein, 130 aa, synthetic construct
  REGION 1..130: note = synthetic sequence
MRAWIFFLLCLAGRALADVVMTQSPLSLPVTLGQPASISCRSSQSIVHSNANTYLEWYQQRPGQSPRLLIYKISNRFSGVPDRFSGSGSGTDFTLKISRVEAEDVGVYYCFQGSHVPRTFGGGTKLEIKR

SEQ ID NO: 201 - protein, 130 aa, synthetic construct
  REGION 1..130: note = synthetic sequence
MRAWIFFLLCLAGRALADVVMTQSPLSLPVTLGQPASISCRSSQSIVHSNDNTYLEWYQQRPGQSPRLLIYKISNRFSGVPDRFSGSGSGTDFTLKISRVEAEDVGVYYCFQGSHVPRTFGGGTKLEIKR

SEQ ID NO: 202 - protein, 130 aa, synthetic construct
  REGION 1..130: note = synthetic sequence
MRAWIFFLLCLAGRALADVVMTQSPLSLPVTLGQPASISCRSSQSIVHSFGNTYLEWYQQRPGQSPRLLIYKISNRFSGVPDRFSGSGSGTDFTLKISRVEAEDVGVYYCFQGSHVPRTFGGGTKLEIKR

SEQ ID NO: 203 - protein, 130 aa, synthetic construct
  REGION 1..130: note = synthetic sequence
MRAWIFFLLCLAGRALADVVMTQSPLSLPVTLGQPASISCRSSQSIVHSNVNTYLEWYQQRPGQSPRLLIYKISNRFSGVPDRFSGSGSGTDFTLKISRVEAEDVGVYYCFQGSHVPRTFGGGTKLEIKR

SEQ ID NO: 204 - protein, 130 aa, synthetic construct; identical to SEQ ID NO: 192
  REGION 1..130: note = synthetic sequence

SEQ ID NO: 205 - protein, 130 aa, synthetic construct; identical to SEQ ID NO: 193
  REGION 1..130: note = synthetic sequence

SEQ ID NO: 206 - protein, 153 aa, synthetic construct
  REGION 1..153: note = synthetic sequence
MEFGLSWVFLVALLRGVQCQVQLVESGGGVVQPGRSLRLSCAASGFTFSSYAMHWVRQAPGKGLEWVAVISYDGSNKYYADSVKGRFTISRDNSKNTLYLQMNSLRADDTAVYYCAKAPGPMVRGVIPDIWGQGTMVTVSSGSASAPTLFPLA

SEQ ID NO: 207 - protein, 153 aa, synthetic construct
  REGION 1..153: note = synthetic sequence
MEFGLSWVFLVALLRGVQCQVQLVESGGGVVQPGRSLRLSCAASGFTFSNYAMHWVRQAPGKGLEWVAVISYDGNNKYYADSVKGRFTISRDNFKNTLYLQMNSLRADDTAVYYCAKAPGSMVRGVIPDIWGQGTMVTVSSGSASAPTLFPLA

SEQ ID NO: 208 - protein, 153 aa, synthetic construct
  REGION 1..153: note = synthetic sequence
MEFGLSWVFLVALLRGVQCQVQLVESGGGVVQPGRSLRLSCAASGFTFSSYVMHWVRQAPGKGLEWVAVISYDGSNKYYADSVKGRFTISRDNSNNTLYLQMNSLRADDTAVYYCAKAPGPMVRGVIPDIWGQGTMVTVSSGSASAPTLFPLA

SEQ ID NO: 209 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFNNTLYLQMNSLRADDTAVYYCAKAPGPMVRGVIPDIWGQGTMVTVSSGSASAPTLFPLA

SEQ ID NO: 210 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFNNTLYLQMNSLRADDTAVYYCAKAPGSMVRGVIPDIWGQGTMVTVSSGSASAPTLFPLA

SEQ ID NO: 211 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRAEDTAVYYCAKAPGPMVRGVIPDIWGQGTMVTVSSGSASAPTLFPLA

SEQ ID NO: 212 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRADDTAVYYCAKAPGSMVRGVIPDIWGQGTMVTVSSGSASAPTLFPLA

SEQ ID NO: 213 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFKNTLYLQMNSLRADDTAVYYCAKAPGSMVRGVIPDIWGQGTMVTVSSGSASAPTLFPLA

SEQ ID NO: 214 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQAPGKGLEWVAVISYDGSNKYYADSVKGRFTISRDNSNNTLYLQMNSLRADDTAVYYCAKAPGPMVRGVIPDIWGQGTMVTVSSGSASAPTLFPLA

SEQ ID NO: 215 - protein, 153 aa, synthetic construct; identical to SEQ ID NO: 214
  REGION 1..153: note = synthetic sequence

SEQ ID NO: 216 - protein, 153 aa, synthetic construct
  REGION 1..153: note = synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRADDTAVYYCAKAPGPMVRGVIPDIWGQGTMVTVSSGSASAPTLFPLA

SEQ ID NO: 217 - protein, 153 aa, synthetic construct; identical to SEQ ID NO: 216
  REGION 1..153: note = synthetic sequence

SEQ ID NO: 218 - protein, 130 aa, synthetic construct
  REGION 1..130: note = synthetic sequence
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQGTHWPPYTFGQGTKLEIKR

SEQ ID NO: 219 - protein, 130 aa, synthetic construct
  REGION 1..130: note = synthetic sequence
METPAQLLFLLLLWLPDTTGEIVLTQSPGTLSLSPGERATLSCRASQSVISSYLAWYQQKPGQAPRLLIYGTSSRATGIPDRFSGSGSGTDFTLTISRLEPEDFAVYYCMQGTHWLPYTFGQGTKLEIKR

SEQ ID NO: 220 - protein, 130 aa, synthetic construct
  REGION 1..130: note = synthetic sequence
METPAQLLFLLLLWLPDTTGEIVLTQSPGTLSLSPGERATLSCRTSQSVRSSYLGWYQQKPGQVPRLLIYGASSRATGIPDRFSGSGSGTDFTLTISRLEPEDFAVYYCMQATHWPPYTFGQGTKLEIKR

SEQ ID NO: 221 - protein, 130 aa, synthetic construct
  REGION 1..130: note = synthetic sequence
METPAQLLFLLLLWLPDTTGEIVLTQSPGTLSLSPGERATLSCRTSQSVRSSYLGWYQQKPGQVPRLLIYGTSIRATGIPDRFSGSGSGTDFTLTISRLEPEDFAVYYCMQATHWPPYTFGQGTKLEIKR

SEQ ID NO: 222 - protein, 147 aa, synthetic construct
  REGION 1..147: note = synthetic sequence
METPAQLLFLLLLWLPDTTGEIVLTQSPGTLSLSPGERATLSCRTSQSVRSSYLGWYQQKPGQVPRLLIYGTSIRATGIPDRFSGSGSGTDFTLTISRLEPEDFAVYYCMQATHWPPYTFGQGTKLEIKRTVAAPSVFIFPPSDEQL

SEQ ID NO: 223 - protein, 130 aa, synthetic construct
  REGION 1..130: note = synthetic sequence
METPAQLLFLLLLWLPDTTGEIVLTQSPGTLSLSPGERATLSCRASQSVISSYLAWYQQKPGQTPRLLIYGTSSRATGIPDRFSGSGSGTDFTLTISRLEPEDFAVYYCMQGTHWLPYTFGQGTKLEIKR

SEQ ID NO: 224 - protein, 130 aa, synthetic construct
  REGION 1..130: note = synthetic sequence
METPAQLLFLLLLWLPDTTGEIVLTQSPGTLSLSPGERATLSCRASQSVISSYLAWYQQKPGQAPRLLIYGTSIRATGIPDRFSGSGSGTDFTLTISRLEPEDFAVYYCMQGTHWLPYTFGQGTKLEIKR

SEQ ID NO: 225 - protein, 130 aa, synthetic construct
  REGION 1..130: note = synthetic sequence
METPAQLLFLLLLWLPDTTGEIVLTQSPGTLSLSPGERATLSCRTSQSVRSSYLAWYQQKPGQAPRLLIYGASSRATGIPDRFSGSGSGTDFTLTISRLEPEDFAVYYCMQATHWPPYTFGQGTKLEIKR

SEQ ID NO: 226 - protein, 130 aa, synthetic construct
  REGION 1..130: note = synthetic sequence
METPAQLLFLLLLWLPDTTGEIVLTQSPGTLSLSPGERATLSCRTSQSVRSSYLGWYQQKPGQAPRLLIYGASSRATGIPDRFSGSGSGTDFTLTISRLEPEDFAVYYCMQATHWPPYTFGQGTKLEIKR

SEQ ID NO: 227 - protein, 130 aa, synthetic construct
  REGION 1..130: note = synthetic sequence
METPAQLLFLLLLWLPDTTGEIVLTQSPGTLSLSPGERATLSCRTSQSVSSSYLGWYQQKPGQAPRLLIYGASSRATGIPDRFSGSGSGTDFTLTISRLEPEDFAVYYCMQATHWPPYTFGQGTKLEIKR

SEQ ID NO: 228 - protein, 130 aa, synthetic construct
  REGION 1..130: note = synthetic sequence
METPAQLLFLLLLWLPDTTGEIVLTQSPGTLSLSPGERATLSCRTSQSVSSSYLGWYQQKPGQVPRLLIYGASSRATGIPDRFSGSGSGTDFTLTISRLEPEDFAVYYCMQATHWPPYTFGQGTKLEIKR

SEQ ID NO: 229 - protein, 130 aa, synthetic construct
  REGION 1..130: note = synthetic sequence
METPAQLLFLLLLWLPDTTGEIVLTQSPGTLSLSPGERATLSCRTSQSVSSSYLAWYQQKPGQVPRLLIYGASSRATGIPDRFSGSGSGTDFTLTISRLEPEDFAVYYCMQATHWPPYTFGQGTKLEIKR

SEQ ID NO: 230 - protein, 130 aa, synthetic construct
  REGION 1..130: note = synthetic sequence
METPAQLLFLLLLWLPDTTGEIVLTQSPGTLSLSPGERATLSCRTSQSVSSSYLAWYQQKPGQAPRLLIYGASSRATGIPDRFSGSGSGTDFTLTISRLEPEDFAVYYCMQATHWPPYTFGQGTKLEIKR

SEQ ID NO: 231 - protein, 130 aa, synthetic construct
  REGION 1..130: note = synthetic sequence
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQATHWPPYTFGQGTKLEIKR

(19) United States
(12) Patent Application Publication
(10) Pub. No.: US 2024/0294631 A1
(43) Pub. Date: Sep. 5, 2024

(54) ANTIBODIES DIRECTED AGAINST INTERLEUKIN-33 (IL-33)

(71) Applicant: AnaptysBio, Inc., San Diego, CA (US)

(72) Inventors: Robert A. Horlick, San Diego, CA (US); David J. King, Encinitas, CA (US); Andrew John McKnight, San Diego, CA (US)

(21) Appl. No.: 18/651,131

(22) Filed: Apr. 30, 2024

Related U.S. Application Data
(63) Continuation of application No. 17/071,046, filed on Oct. 15, 2020, now Pat. No. 11,999,783.
Application No. 17/071,046 is a continuation of application No. 16/042,476, filed on Jul. 23, 2018, now Pat. No. 10,836,820.
Application No. 16/042,476 is a division of application No. 15/110,724, filed on Jul. 8, 2016, now Pat. No. 10,059,764.
(60) Provisional application No. 61/925,946, filed on Jan. 10, 2014.

(51) Int. Cl. C07K 16/24

(52) U.S. Cl. CPC C07K 16/244; C07K 2317/56; C07K 2317/565; C07K 2317/76; C07K 2317/92

(57) ABSTRACT

The invention relates to an isolated immunoglobulin heavy chain polypeptide and an isolated immunoglobulin light chain polypeptide that bind to interleukin-33 (IL-33). The invention provides an IL-33-binding agent that comprises the aforementioned immunoglobulin heavy chain polypeptide and immunoglobulin light chain polypeptide. The invention also provides related vectors, compositions, and methods of using the IL-33-binding agent to treat a disorder in a mammal that is responsive to IL-33 inhibition.

### CROSS-REFERENCE TO RELATED APPLICATIONS

This patent application is a continuation of co-pending U.S. patent application Ser. No. 17/071,046, filed on Oct. 15, 2020, which is a continuation of co-pending U.S. patent application Ser. No. 16/042,476, filed on Jul. 23, 2018, now U.S. Pat. No. 10,836,820, which is a divisional of U.S. patent application Ser. No. 15/110,724, filed on Jul. 8, 2016, now U.S. Pat. No. 10,059,764, which is the U.S. national phase of International Patent Application No. PCT/US2015/010785, filed on Jan. 9, 2015, which claims the benefit of U.S. Patent Application No. 61/925,946, filed on Jan. 10, 2014, each of which are incorporated by reference in their entireties herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 317,102 Byte XML file named “770929_ST26.xml,” created on Apr. 24, 2024.

### BACKGROUND OF THE INVENTION

Interleukin 33 (IL-33), also known as nuclear factor (NF) in high endothelial venules (NF-HEV), is a cytokine belonging to the IL-1 superfamily. IL-33 induces helper T cells, mast cells, eosinophils and basophils to produce type 2 cytokines. IL-33 mediates its biological effects by interacting with the receptors ST2 (also known as ILIRL1) and IL-1 Receptor Accessory Protein (IL1RAP) to activate intracellular molecules in the NF-κB and MAP kinase signaling pathways that drive production of type 2 cytokines (e.g., IL-4, IL-5, and IL-13) from polarized helper T cells (Th2) and Group-2 innate lymphoid cells (ILC2) in the skin, lungs, and gastrointestinal tract. IL-33 acts directly on mast cells to trigger their activation, and stimulates eosinophils and basophils to degranulate, causing tissue damage. The induction of type 2 cytokines by IL-33 in vivo is believed to induce the severe pathological changes observed in mucosal organs following administration of IL-33 (see, e.g., Schmitz et al., Immunity, 23(5): 479-490 (2005); and Chackerian et al., J. Immunol., 179 (4): 2551-2555 (2007))

Both the in vivo expression profile of IL-33 and its cellular targets suggest a role for IL-33 in Th2-driven pathologies. For example, IL-33 expression has been detected in inflamed tissue from patients with moderate-to-severe asthma, atopic dermatitis, allergic rhinitis, food allergies, rheumatoid arthritis, multiple sclerosis, and Crohn's disease. In addition, functional single nucleotide polymorphisms (SNPs) in the distal promoter region of ST2 (IL-33R) have shown a significant association with atopic dermatitis (see, e.g., Shimizu et al., Hum. Mol. Genet., 14(19): 2919-2927 (2005)). Genome-wide association studies (GWAS) have also shown a strong link with SNPs in IL-33 and ST2 (IL-33R) genes for asthma in multiple studies of ethnically diverse groups (see, e.g., Gudbjartsson et al., Nat. Genet., 41(3): 342-347 (2009); Melen et al., J Allergy Clin. Immunol., 126(3): 631-637 (2010); Moffatt et al., New Engl J. Med., 363(13): 1211-1221 (2010); and Torgerson et al., Nat. Genet., 43(9): 887-92 (2011)). IL-33 (possibly in combination with IL-25 and TSLP) also activates innate lymphoid cells (ILC2 cells) leading to Th2 cytokine secretion, anti-parasitic responses, and tissue immunopathology.

Studies also suggest that IL-33 plays a direct role in some cancers expressing the IL-33 receptor such as, for example, epithelial cancers (i.e., carcinomas) by acting as a survival or growth factor for cancer cells. Such responsiveness to IL-33 might contribute to escape of certain cancer cell types from current standard of care (e.g., chronic myelogenous leukemia (CML), breast cancers, and gastrointestinal cancers). Furthermore, IL-33 may play an indirect role in cancer progression by reducing the protective activity of the immune system in controlling tumor cells. Other recent studies suggest that IL-33 plays a role in the pathology of fibrosis, such as, for example, skin fibrosis, liver fibrosis, systemic sclerosis, and lung fibrosis. In addition, Mchedlidze et al., Immunity, 39: 357-371 (2013), demonstrates that hepatic expression of interleukin-33 (IL-33) is both required and sufficient for severe hepatic fibrosis in vivo.

Therefore, there is a need for inhibitors of IL-33 (e.g., antibodies) that bind IL-33 with high affinity and effectively neutralize IL-33 activity. The invention provides an IL-33 binding agent that binds to and inhibits IL-33.

### BRIEF SUMMARY OF THE INVENTION

The invention provides an isolated immunoglobulin heavy chain polypeptide which comprises (a) an amino acid sequence of any one of SEQ ID NO: 1, SEQ ID NO: 2, and SEQ ID NOs: 5-50, or (b) an amino acid sequence that is at least 90% identical to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217.

The invention provides an isolated immunoglobulin light chain polypeptide which comprises (a) an amino acid sequence of any one of SEQ ID NO: 3, SEQ ID NO: 4, and SEQ ID NOs: 51-66, or (b) an amino acid sequence that is at least 90% identical to any one of SEQ ID NO: 3, SEQ ID NO: 4, SEQ ID NOs: 51-66, SEQ ID NOs: 141-175, SEQ ID NOs: 189-205, and SEQ ID NOs: 218-231.

In addition, the invention provides isolated or purified nucleic acid sequences encoding the foregoing immunoglobulin polypeptides, vectors comprising such nucleic acid sequences, isolated IL-33-binding agents comprising the foregoing immunoglobulin polypeptides, nucleic acid sequences encoding such IL-33-binding agents, vectors comprising such nucleic acid sequences, isolated cells comprising such vectors, compositions comprising such IL-33-binding agents or such vectors with a pharmaceutically acceptable carrier, and methods of treating a disease or disorder in mammals that is responsive to IL-33 inhibition or neutralization by administering effective amounts of such compositions to mammals.

### BRIEF DESCRIPTION OF THE SEVERAL VIEW OF THE DRAWINGS

FIG. 1A is a graph which depicts experimental data illustrating the determination of EC₅₀ for IL-33 stimulation of IL-5 secretion from KU812 cells. FIG. 1B is a graph which depicts experimental data illustrating that the inventive IL-33 binding agent inhibits IL-33-mediated release of IL-5 from KU812 cells.

FIG. 2A is a graph which depicts experimental data illustrating that IL-33-induces expression of luciferase from the IL-8 promoter in HEK293-ST2 cells. FIG. 2B is a graph which depicts experimental data illustrating that the inventive IL-33 binding agent inhibits IL-33-induced expression of luciferase from the IL-8 promoter in HEK293-ST2 cells.

FIG. 3 is a graph which depicts experimental data illustrating that the inventive IL-33-binding agent inhibits IL-33-mediated release of IL-5 in primary human basophils. For APE4909, IC50=2.2±1.1 nM (N=3); for ST2 monomer, IC50=20 nM (N=1). The dashed lines marked “IL-33” and “medium” represent the concentration of IL-5 secreted in the absence of antibody and in the absence of IL-33, respectively. The 0.02 suffix appended to APE4909 refers to the lot of protein tested in these experiments.

FIG. 4 is a graph which depicts experimental data illustrating that the inventive IL-33-binding agent inhibits IL-33-mediated release of IL-9 from primary human basophils. The IC50 for APE4909 was measured at 3 nM. The dashed lines marked “IL-33” and “medium” represent the concentration of IL-9 secreted in the absence of antibody and in the absence of IL-33, respectively. The antibody APE0422 represents an isotype control antibody.

FIG. 5 is a graph which depicts experimental data illustrating affinity of the inventive APE4909 antibody for human IL-33 as measured by KINEXA™. Results indicate KD=1.0 pM (N=2) with 95% confidence interval (CI) of 1.9 pM-420 fM.

FIG. 6 is a graph which depicts experimental data illustrating affinity of the inventive APE4909 antibody for cynomolgus IL-33 as measured by KINEXA™.

FIG. 7 is a graph which depicts experimental data illustrating the ability of the inventive APE4909 antibody to inhibit human IL-33-driven cosinophil expansion in the peripheral blood compartment.

### DETAILED DESCRIPTION OF THE INVENTION

The invention provides an isolated immunoglobulin heavy chain polypeptide and/or an isolated immunoglobulin light chain polypeptide, or a fragment (e.g., antigen-binding fragment) thereof. The term “immunoglobulin” or “antibody,” as used herein, refers to a protein that is found in blood or other bodily fluids of vertebrates, which is used by the immune system to identify and neutralize foreign objects, such as bacteria and viruses. The polypeptide is “isolated” in that it is removed from its natural environment. In a preferred embodiment, an immunoglobulin or antibody is a protein that comprises at least one complementarity determining region (CDR). The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, NY (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The isolated immunoglobulin heavy chain polypeptide and the isolated immunoglobulin light chain polypeptide of the invention desirably bind to IL-33. As discussed above, interleukin-33 (IL-33) (also known as nuclear factor (NF) in high endothelial venules (NF-HEV)) is a cytokine of the IL-1 family, which also includes the inflammatory cytokines IL-1α, IL-1β, and IL-18. IL-33 has been shown to signal via the ST2 receptor and the IL1RAP receptor. IL-33 is expressed broadly in various tissues, including stomach, lung, spinal cord, brain, and skin, as well as in cells, including smooth muscle cells and epithelial cells lining bronchus and small airways. IL-33 expression is induced by IL-1β and tumor necrosis factor-α (TNF-α) in lung and dermal fibroblasts and, to a lesser extent, by macrophage activation. IL-33 treatment has been shown to induce T-helper (Th) type 2 responses in mice as indicated by an increase in Th2 cytokine production and serum immunoglobulin. Systemic treatment of mice with IL-33 results in pathologic changes in the lung and the digestive tract (see, e.g., Choi et al., Blood, 114(14): 3117-3126 (2009); and Yagami et al., J. Immunology, 185(10): 5743-5750 (2010)).

IL-33 is produced as a 30-kDa precursor protein that is cleaved in vitro by caspase-1, releasing the mature 18-kDa form (see, e.g., Schmitz et al., Immunity, 23(5): 479-490(2005)). Upon binding to the ST2 receptor, IL-33 promotes the activation of nuclear factor (NF)-κB and mitogen-activated protein kinase (MAPK), leading to increased transcription of Th2 cytokines (Schmitz et al., supra).

Antibodies which bind to IL-33, and components thereof, are known in the art (see, e.g., U.S. Patent Application Publications 2009/0041718 A1 and 2012/0263709 A1). Anti-IL-33 antibodies also are commercially available from sources such as, for example, Abcam (Cambridge, MA).

The invention provides an immunoglobulin heavy chain polypeptide that comprises an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOS: 206-217, or an amino acid sequence that is at least 90% identical to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217. In one embodiment of the invention, the isolated immunoglobulin heavy chain polypeptide comprises, consists of, or consists essentially of an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217. When the inventive immunoglobulin heavy chain polypeptide consists essentially of an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, additional components can be included in the polypeptide that do not materially affect the polypeptide (e.g., protein moieties such as biotin that facilitate purification or isolation). When the inventive immunoglobulin heavy chain polypeptide consists of an amino acid sequence of any one of SEQ ID NO: 1, SEQ ID NO: 2, SEQ ID NOs: 5-50, SEQ ID NOS: 67-140, SEQ ID NO: 176, SEQ ID NO: 177, SEQ ID NOs: 178-188, and SEQ ID NOs: 206-217, the polypeptide does not comprise any additional components (i.e., components that are not endogenous to the inventive immunoglobulin heavy chain polypeptide).

The invention provides an isolated immunoglobulin heavy chain polypeptide which comprises an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, or SEQ ID NOs: 206-217. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The invention provides an immunoglobulin light chain polypeptide that comprises an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, or an amino acid sequence that is at least 90% identical to any one of SEQ ID NO: 3, SEQ ID NO: 4, SEQ ID NOs: 51-66, SEQ ID NOs: 141-175, SEQ ID NOs: 189-205, and SEQ ID NOs: 218-231. In one embodiment of the invention, the isolated immunoglobulin light chain polypeptide comprises, consists of, or consists essentially of an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231. When the inventive immunoglobulin light chain polypeptide consists essentially of an amino acid sequence of any one of SEQ ID NO: 3, SEQ ID NO: 4, SEQ ID NOs: 51-66, SEQ ID NOS: 141-175, SEQ ID NOs: 189-205, and SEQ ID NOs: 218-231, additional components can be included in the polypeptide that do not materially affect the polypeptide (e.g., protein moieties such as biotin that facilitate purification or isolation). When the inventive immunoglobulin light chain polypeptide consists of an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, the polypeptide does not comprise any additional components (i.e., components that are not endogenous to the inventive immunoglobulin light chain polypeptide).

The invention provides an isolated immunoglobulin light chain polypeptide which comprises an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NO: 3, SEQ ID NO: 4, SEQ ID NOs: 51-66, SEQ ID NOs: 141-175, SEQ ID NOs: 189-205, or SEQ ID NOs: 218-231. Nucleic acid or amino acid sequence “identity,” as described herein, can be determined using the methods described herein.

One or more amino acids of the aforementioned immunoglobulin heavy chain polypeptides and/or light chain polypeptides can be replaced or substituted with a different amino acid. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free-NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the aforementioned immunoglobulin heavy chain polypeptides and/or light chain polypeptides. Any number of any suitable amino acids can be inserted into the amino acid sequence of the immunoglobulin heavy chain polypeptide and/or light chain polypeptide. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy chain polypeptide and/or light chain polypeptide. Preferably, 1-10 amino acids (e.g., 1, 2, 3, 4, 5, 6, 7, 8, 9, or 10 amino acids) are inserted into the amino acid sequence of the immunoglobulin heavy chain polypeptide and/or light chain polypeptide. In this respect, the amino acid(s) can be inserted into any one of the aforementioned immunoglobulin heavy chain polypeptides and/or light chain polypeptides in any suitable location. Preferably, the amino acid(s) are inserted into a CDR (e.g., CDR1, CDR2, or CDR3) of the immunoglobulin heavy chain polypeptide and/or light chain polypeptide.

The inventive isolated immunoglobulin heavy chain polypeptide and light chain polypeptides are not limited to polypeptides comprising the specific amino acid sequences described herein. Indeed, the immunoglobulin heavy chain polypeptide or light chain polypeptide can be any heavy chain polypeptide or light chain polypeptide that competes with the inventive immunoglobulin heavy chain polypeptide or light chain polypeptide for binding to IL-33. In this respect, for example, the immunoglobulin heavy chain polypeptide or light chain polypeptide can be any heavy chain polypeptide or light chain polypeptide that binds to the same epitope of IL-33 recognized by the heavy and light chain polypeptides described herein. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The invention provides an isolated interleukin-33 (IL-33)-binding agent comprising, consisting essentially of, or consisting of one or more of the inventive isolated amino acid sequences described herein. By “interleukin-33 (IL-33)-binding agent” is meant a molecule, preferably a proteinaccous molecule, that binds specifically to IL-33. Preferably, the IL-33-binding agent is an antibody or a fragment (e.g., immunogenic fragment) thereof. The isolated IL-33-binding agent of the invention comprises, consists essentially of, or consists of the inventive isolated immunoglobulin heavy chain polypeptide and/or the inventive isolated immunoglobulin light chain polypeptide. In one embodiment, the isolated IL-33-binding agent comprises, consists essentially of, or consists of the inventive immunoglobulin heavy chain polypeptide or the inventive immunoglobulin light chain polypeptide. In another embodiment, the isolated IL-33-binding agent comprises, consists essentially of, or consists of the inventive immunoglobulin heavy chain polypeptide and the inventive immunoglobulin light chain polypeptide.

Any amino acid residue of the inventive immunoglobulin heavy chain polypeptide and/or the inventive immunoglobulin light chain polypeptide can be replaced, in any combination, with a different amino acid residue, or can be deleted or inserted, so long as the biological activity of the IL-33-binding agent is enhanced or improved as a result of the amino acid replacements, insertions, and/or deletions. The “biological activity” of an IL-33-binding agent refers to, for example, binding affinity for a particular IL-33 epitope, neutralization or inhibition of IL-33 binding to its receptor(s), neutralization or inhibition of IL-33 activity in vivo (e.g., IC50), pharmacokinetics, and cross-reactivity (e.g., with non-human homologs or orthologs of the IL-33 protein, or with other proteins or tissues). Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The terms “inhibit” or “neutralize,” as used herein with respect to the activity of a IL-33-binding agent, refer to the ability to substantially antagonize, prohibit, prevent, restrain, slow, disrupt, alter, eliminate, stop, or reverse the progression or severity of, for example, the biological activity of IL-33, or a disease or condition associated with IL-33. The isolated IL-33-binding agent of the invention preferably inhibits or neutralizes the activity of IL-33 by at least about 20%, about 30%, about 40%, about 50%, about 60%, about 70%, about 80%, about 90%, about 95%, about 100%, or a range defined by any two of the foregoing values.

The isolated IL-33-binding agent of the invention can be a whole antibody, as described herein, or an antibody fragment. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The isolated IL-33 binding agent can contain any IL-33-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (VH or VL) polypeptide that specifically binds antigen.

In embodiments where the isolated IL-33-binding agent comprises a fragment of the immunoglobulin heavy chain or light chain polypeptide, the fragment can be of any size so long as the fragment binds to, and preferably inhibits the activity of, IL-33. In this respect, a fragment of the immunoglobulin heavy chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids. Similarly, a fragment of the immunoglobulin light chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids.

When the IL-33-binding agent is an antibody or antibody fragment, the antibody or antibody fragment desirably comprises a heavy chain constant region (Fe) of any suitable class. Preferably, the antibody or antibody fragment comprises a heavy chain constant region that is based upon wild-type IgG1, IgG2, or IgG4 antibodies, or variants thereof.

The IL-33-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The isolated IL-33-binding agent also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The isolated IL-33-binding agent also can be an antibody conjugate. In this respect, the isolated IL-33-binding agent can be a conjugate of (1) an antibody, an alternative scaffold, or fragments thereof, and (2) a protein or non-protein moiety comprising the IL-33-binding agent. For example, the IL-33-binding agent can be all or part of an antibody conjugated to a peptide, a fluorescent molecule, or a chemotherapeutic agent.

The isolated IL-33-binding agent can be, or can be obtained from, a human antibody, a non-human antibody, or a chimeric antibody. By “chimeric” is meant an antibody or fragment thereof comprising both human and non-human regions. Preferably, the isolated IL-33-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the inventive IL-33-binding agent is obtained or derived from a mouse monoclonal antibody, while the remaining variable regions and constant region of the inventive IL-33-binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

In one embodiment, a CDR (e.g., CDR1, CDR2, or CDR3) or a variable region of the immunoglobulin heavy chain polypeptide and/or the immunoglobulin light chain polypeptide described herein can be transplanted (i.e., grafted) into another molecule, such as an antibody or non-antibody polypeptide, using either protein chemistry or recombinant DNA technology. In this regard, the invention provides an isolated IL-33-binding agent comprising at least one CDR of an immunoglobulin heavy chain and/or light chain polypeptide as described herein. The isolated IL-33-binding agent can comprise one, two, or three CDRs of an immunoglobulin heavy chain and/or light chain variable region as described herein. For example, with respect to immunoglobulin heavy chain polypeptides comprising any one of SEQ ID NO: 1, SEQ ID NO: 2, or SEQ ID NOs: 5-50, the CDR1 is located between amino acid residues 26 and 35, inclusive; the CDR2 is located between amino acid residues 50 and 59, inclusive (SEQ ID NO: 1 and SEQ ID NO: 2) or between amino acid residues 50 and 66, inclusive (SEQ ID NOs: 5-50); and the CDR3 is located between amino acid residues 99 and 102, inclusive (SEQ ID NO: 1 and SEQ ID NO: 2) or between amino acid residues 99 and 111, inclusive (SEQ ID NOs 5-50). With respect to immunoglobulin light chain polypeptides comprising any one of SEQ ID NO: 3, SEQ ID NO: 4, and SEQ ID NO: 51-66, for example, the CDR1 is located between amino acid residues 24 and 39, inclusive (SEQ ID NO: 3 and SEQ ID NO: 4) or between amino acid residues 24 and 34, inclusive (SEQ ID NOs: 51-66); the CDR2 is located between amino acid residues 55 and 61, inclusive (SEQ ID NO: 3 and SEQ ID NO: 4) or between amino acid residues 50 and 56, inclusive (SEQ ID NOs: 51-66); the CDR3 is located between amino acid residues 94 and 102, inclusive (SEQ ID NO: 3 and SEQ ID NO: 4) or between amino acid residues 89 and 97, inclusive (SEQ ID NOs: 51-66).

In a preferred embodiment, the IL-33-binding agent binds an epitope of IL-33 which blocks the binding of IL-33 to receptors ST2 (also known as ILIRL1) and/or IL-1 Receptor Accessory Protein (IL1RAP) and inhibits IL-33 mediated signaling. The invention also provides an isolated or purified epitope of IL-33 which blocks the binding of IL-33 to receptors ST2 and IL1RAP in an indirect or allosteric manner.

The invention also provides one or more isolated or purified nucleic acid sequences that encode the inventive immunoglobulin heavy chain polypeptide, the inventive immunoglobulin light chain polypeptide, and the inventive IL-33-binding agent.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The invention further provides a vector comprising one or more nucleic acid sequences encoding the inventive immunoglobulin heavy chain polypeptide, the inventive immunoglobulin light chain polypeptide, and/or the inventive IL-33-binding agent. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In addition to the nucleic acid sequence encoding the inventive immunoglobulin heavy polypeptide, the inventive immunoglobulin light chain polypeptide, and/or the inventive IL-33-binding agent, the vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ system (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pBK-CMV from Stratagene (La Jolla, CA) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Life Technologies (Carlsbad, CA), UCOE from Millipore (Billerica, MA), and pCI or pFN10A (ACT) FLEXI™ from Promega (Madison, WI).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, CA), and the retroviral vectors pFB-ERV plus pCFB-EGSH from Stratagene (La Jolla, CA).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially.

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an isolated cell comprising the inventive vector. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5a, DH10, MC1061 (ATCC No. 53338), and CC102).

Preferably, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, CA).

Preferably, mammalian cells are utilized in the invention. A number of suitable mammalian host cells are known in the art, and many are available from the American Type Culture Collection (ATCC, Manassas, VA). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

Most preferably, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

A nucleic acid sequence encoding the inventive amino acid sequence may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

The invention provides a composition comprising an effective amount of the inventive immunoglobulin heavy chain polypeptide, the inventive immunoglobulin light chain polypeptide, the inventive IL-33-binding agent, the inventive nucleic acid sequence encoding any of the foregoing, or the inventive vector comprising the inventive nucleic acid sequence. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The invention further provides a method of treating a disease or disorder in a mammal that is responsive to IL-33 inhibition or neutralization. The method comprises administering the aforementioned composition to a mammal having a disease or disorder that is responsive to IL-33 inhibition or neutralization, whereupon the disease disorder is treated in the mammal. A disease or disorder that is “responsive to IL-33 inhibition” or “responsive to IL-33 neutralization,” refers to any disease or disorder in which a decrease in IL-33 levels or activity has a therapeutic benefit in mammals, preferably humans, or the improper expression (e.g., overexpression) or increased activity of IL-33 causes or contributes to the pathological effects of the disease or disorder. Diseases or disorders that are responsive to IL-33 inhibition or neutralization include, for example, inflammatory disorders, autoimmune diseases, certain cancers (e.g., epithelial cancers (carcinomas), chronic myelogenous leukemia (CML), breast cancers, and gastrointestinal cancers), and any atopic disorder. Inflammatory disorders include, for example, allergic inflammation of the skin, lungs, and gastrointestinal tract, atopic dermatitis (also known as atopic eczema), asthma (allergic and non-allergic), fibrosis (e.g., idiopathic pulmonary fibrosis, scleroderma, kidney fibrosis, and scarring), chronic obstructive pulmonary disease (COPD), allergic rhinitis, food allergies (e.g., allergies to peanuts, eggs, dairy, shellfish, tree nuts, etc.), seasonal allergies, and other allergies. Autoimmune diseases include, for example, Crohn's disease, rheumatoid arthritis, psoriasis, ankylosing spondylitis, lupus erythematosus, and scleroderma. The term “atopic,” as used herein, refers to a hereditary predisposition toward developing certain hypersensitivity reactions (e.g., eczema (atopic dermatitis), hay fever (allergic rhinitis), and allergy-induced asthma (allergic asthma)), which is typically mediated by excessive IgE production.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the IL-33-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the IL-33-binding agent to elicit a desired response in the individual. For example, a therapeutically effective amount of an IL-33-binding agent of the invention is an amount which decreases IL-33 bioactivity in a human.

Alternatively, the pharmacologic and/or physiologic effect may be prophylactic, i.e., the effect completely or partially prevents a disease or symptom thereof. In this respect, the inventive method comprises administering a “prophylactically effective amount” of the IL-33-binding agent. A “prophylactically effective amount” refers to an amount effective, at dosages and for periods of time necessary, to achieve a desired prophylactic result (e.g., prevention of disease onset).

A typical dose can be, for example, in the range of 1 μg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The composition comprising an effective amount of the inventive immunoglobulin heavy chain polypeptide, the inventive immunoglobulin light chain polypeptide, the inventive IL-33-binding agent, the inventive nucleic acid sequence encoding any of the foregoing, or the inventive vector comprising the inventive nucleic acid sequence can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a cross-reactive human), the biological activity of the inventive IL-33-binding agent can be measured by any suitable method known in the art. For example, the biological activity can be assessed by determining the stability of a particular IL-33-binding agent. In one embodiment of the invention, the IL-33-binding agent (e.g., an antibody) has an in vivo half life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the IL-33-binding agent has an in vivo half life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the IL-33-binding agent has an in vivo half life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The biological activity of a particular IL-33-binding agent also can be assessed by determining its binding affinity to IL-33 or an epitope thereof. The term “affinity” refers to the equilibrium constant for the reversible binding of two agents and is expressed as the dissociation constant (KD). Affinity of a binding agent to a ligand, such as affinity of an antibody for an epitope, can be, for example, from about 1 femtomolar (fM) to about 100 micromolar (μM) (e.g., from about 1 fM to about 1 picomolar (pM), from about 1 pM to about 1 nanomolar (nM), from about 1 nM to about 1 micromolar (μM), or from about 1 μM to about 100 pM). In one embodiment, the IL-33-binding agent can bind to an IL-33 protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the IL-33-binding agent can bind to IL-33 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, and/or ELISA (see, e.g., Janeway et al. (eds.), Immunobiology, 5th ed., Garland Publishing, New York, NY, 2001).

The IL-33-binding agent of the invention may be administered alone or in combination with other drugs (e.g., as an adjuvant). For example, the IL-33-binding agent can be administered in combination with other agents for the treatment or prevention of the diseases or disorders disclosed herein. In this respect, the IL-33-binding agent can be used in combination with at least one other anti-inflammatory agent including, for example, corticosteroids (e.g., prednisone and fluticasone) and non-steroidal anti-inflammatory drugs (NSAIDs) (e.g., aspirin, ibuprofen, and naproxen).

In addition to therapeutic uses, the IL-33-binding agent described herein can be used in diagnostic or research applications. In this respect, the IL-33-binding agent can be used in a method to diagnose a disease or disorder that is responsive to IL-33 inhibition or neutralization. In a similar manner, the IL-33-binding agent can be used in an assay to monitor IL-33 protein levels in a subject being tested for a disease or disorder that is responsive to IL-33 inhibition or neutralization. Research applications include, for example, methods that utilize the IL-33-binding agent and a label to detect an IL-33 protein in a sample, e.g., in a human body fluid or in a cell or tissue extract. The IL-33-binding agent can be used with or without modification, such as covalent or non-covalent labeling with a detectable moiety. For example, the detectable moiety can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)).

IL-33 protein levels can be measured using the inventive IL-33-binding agent by any suitable method known in the art. Such methods include, for example, radioimmunoassay (RIA), and FACS. Normal or standard expression values of IL-33 can be established using any suitable technique, e.g., by combining a sample comprising, or suspected of comprising, IL-33 with a IL-33-specific antibody under conditions suitable to form an antigen-antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)). The amount of IL-33 polypeptide expressed in a sample is then compared with a standard value.

The IL-33-binding agent can be provided in a kit, i.e., a packaged combination of reagents in predetermined amounts with instructions for performing a diagnostic assay. If the IL-33-binding agent is labeled with an enzyme, the kit desirably includes substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following examples further illustrate the invention but, of course, should not be construed as in any way limiting its scope.

### Example 1

This example describes assays used to determine the functional activity of the inventive immunoglobulin heavy and light chain polypeptides.

IL-33-Mediated Release of IL5 from KU812 Cells.

KU812 cells, a human basophil-like CML cell line (ATCC No. CRL-2099) (see, e.g., Tare et al., Exp. Cell Res., 316(15): 2527-37 (2010); Lefrancais et al., Proc. Natl. Acad. Sci. USA, 109(5): 1673-1978 (2012)), respond to IL-33 stimulation by secreting IL-5. KU812 cells were suspended in RPM1+10% FBS culture medium, and 500,000 cells per well were plated into 96-well flat bottom plates. A 30 μg/mL stock for antibody of interest was serially diluted to generate 8 concentrations at half-log intervals. The diluted samples were added in rows to cells and incubated at 37° C. for 30 minutes. IL-33-his6-bio (C-terminally labeled with 6 His, and biotinylated with an average of 1 to 2 biotins per molecule) (3 ng) was then added to each well, and plates were incubated at 37° C. for 48 hours. Supernatants were then removed and held at 4° C. until testing by ELISA. Supernatants were tested using an IL-5 DuoSet ELISA kit (R&D Systems, Minneapolis, MN) and evaluated on a SPECTRAMAX™ microplate reader (Molecular Devices, LLC, Sunnyvale, CA) using SOFTMAX PRO™ Microplate Data Acquisition & Analysis Software (Molecular Devices, LLC, Sunnyvale, CA) to determine IL-5 production.

### IL-33-Mediated Expression of Luciferase in HEK293-ST2 Cells

An HEK/ST2-stable cell line was generated by first plating naïve HEK cells at 3×10³ cells/T75 flask in DMEM/10% FBS and incubating overnight at 37° C. The following day, cells were transfected by mixing 500 μL Optimem (Life Technologies, Carlsbad, CA)+24 μL HD FUGENE™ (Promega, Madison, WI) and allowed to incubate at room temperature for five minutes. DNA encoding ST2-Fc (4 μg) was added to the FUGENE™ mixture and allowed to incubate at room temperature for 25 minutes. The DNA/FUGENE™ mixture was then distributed over the HEK cells and allowed to incubate overnight at 37° C., 5% CO₂. At 24 hours post transfection, cells were split and placed under hygromycin selection for a period of 3-4 weeks until stably selected.

### IL-8 Luciferase Reporter Assay

4×10⁶ HEK293/ST2-Fc cells were seeded in a T-75 flask overnight at 37° C., 5% CO₂. The following morning, the DNA construct AB4111 which encodes the human IL-8 promoter driving expression of a luciferase reporter gene, was transfected into the cells by mixing 500 μL Optimem+24 μL HD FUGENE™ and allowed to incubate at room temperature for five minutes. The IL-8 promoter responds to the signal transduction cascade initiated by stimulation of the ST2-IL-IRAcP receptor complex by IL-33 occupancy. AB4111 (2 μg) was added to the FUGENE™ mixture and allowed to incubate at room temperature for 25 minutes. The DNA/FUGENE™ mixture was then distributed over the HEK/ST2 cells and allowed to incubate for 8 hours. Cells were harvested with ACCUTASE™ and seeded into a 96-well, flat bottom plate, with 2.0×10⁴ cells per well in 0.1 mL DMEM/10% FBS. Plates were incubated for 15-18 hours at 37° C., 5% CO₂. The next morning, plates were gently inverted and tapped on paper towels to remove media. 50 μL/well of fresh DMEM/10% FBS was added to each well. Cells were stimulated with pre-complexed IL-33/ST2-Fc or IL-33/Ab for 20 minutes at room temperature and then added to the cells and allowed to incubate for an additional 5 hours at 37° C. After 5 hours, luciferase activity was determined using the Steady Glo-Luciferase Assay System (Promega, Madison, WI) by adding luciferase reagent at 1:1 vol/vol to each well. Wells were mixed and 150 μL/well was transferred to black-walled, clear-bottom plates and read on the ENVISTION™ Plate Reader (PerkinElmer, Waltham, MA) using the Luminescence program (60-sec delay). Data was analyzed using a 4 parameter curve fit with GraphPad Prism 5 software (GraphPad, San Diego, CA).

### Surface Plasmon Resonance (SPR) Methods

Binding kinetics and affinities of anti-IL33 antibodies were determined by SPR on a BIACORE™ T200 instrument (GE Healthcare). Each of four flow cells on a Series S CM5 chip was immobilized with ˜ 10,000 RU anti-human IgG (Fc). Antibodies (˜1 μg/mL) were captured for 60 seconds at a flow rate of 10 μL/min. Monomeric IL-33 was diluted in running buffer (HBS-EP+, pH 7.6) starting at approximately 100-fold higher concentration than each antibody's KD. Each IL-33 concentration was passed over all flow cells for 180 seconds at 30 μL/min, then allowed to dissociate for 1800 seconds. Surfaces were regenerated with 3 M MgCl₂ for 60 seconds. Association and dissociation kinetic constants (kₒₙ and k_{off}) and steady-state affinity (KD) were derived from the resulting sensorgrams using BIACORE T200 Evaluation Software version 1.0.

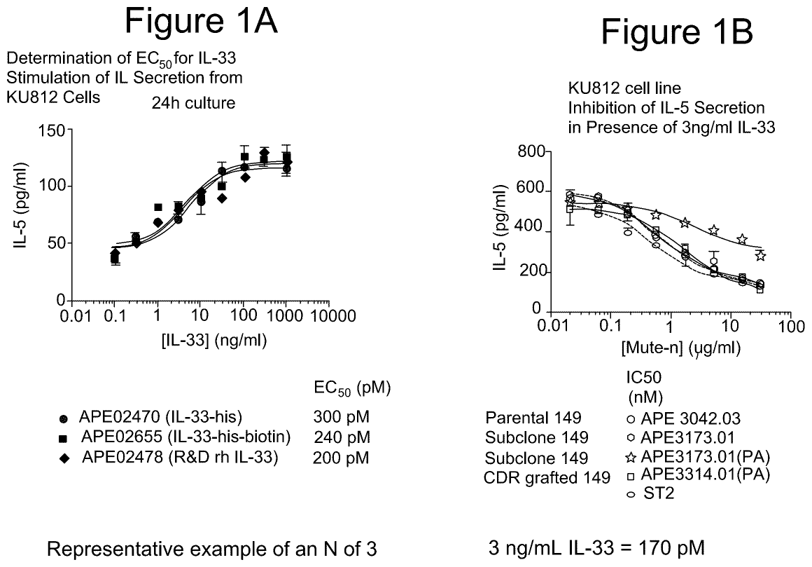

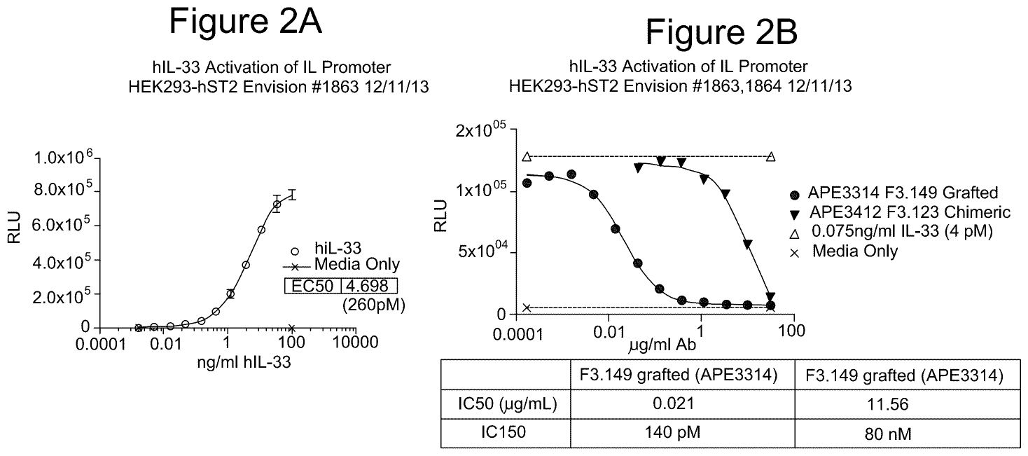

The results of the above assays with respect to several of the IL-33-binding agents described herein are shown in FIGS. 1A, 1B, 2A, and 2B.

### Example 2

This example describes experiments demonstrating the functional activity of an inventive IL-33-binding agent.

An immunoglobulin heavy chain polypeptide comprising the amino acid sequence of SEQ ID NO: 136 was paired with an immunoglobulin light chain polypeptide comprising the amino acid sequence of SEQ ID NO: 171. The resulting antibody was referred to as APE4909. The ability of APE4909 to inhibit IL-33-mediated release of IL-5 and IL-9 in primary human basophils was assessed as described below.

Leukocyte Reduction System (LRS) units processed from donor whole blood were obtained from the San Diego Blood Bank. Peripheral blood mononuclear cells (PBMCs) were prepped by standard methods using Ficoll density centrifugation separation. Approximately 109 PBMCs were typically obtained from an LRS unit. Basophils were isolated from PBMCs using a human basophil isolation kit II (Miltenyi Biotec cat #130-092-662, San Diego, CA). The total yield of basophils was approximately 106.

Basophils were diluted to a density of 2×10⁶/mL in RPMI 1640 medium containing 10% fetal bovine serum, penicillin/streptomycin (P/S), and 25 ng/ml of recombinant human IL-3 (R&D Systems, Minneapolis, MN). 100 μL of diluted cells per well were plated in standard flat-bottom 96-well tissue culture plates for a final cell density of 2×10⁵ per well. Outside wells were filled with 200 μL PBS/well to minimize the effects of non-uniform evaporation. Cells were cultured overnight in 5% CO₂ in a 37° C. incubator.

The following day, APE4909 and a monomeric human ST2 protein (hST2—referred to as APE3906) were added at concentrations ranging from 30 to 0 μg/mL serially diluted at half-log intervals in RPMI+10% FBS+P/S containing 50 ng/ml IL-33. Approximately 18 hours later, plates were centrifuged at 300×g for 3 minutes. Supernatants were removed, transferred to a clean plate, and stored at −80° C. pending analysis.

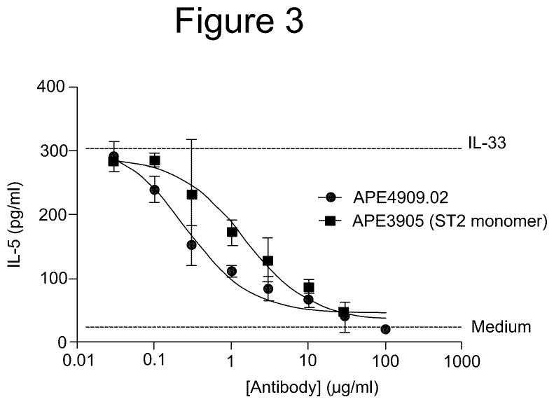

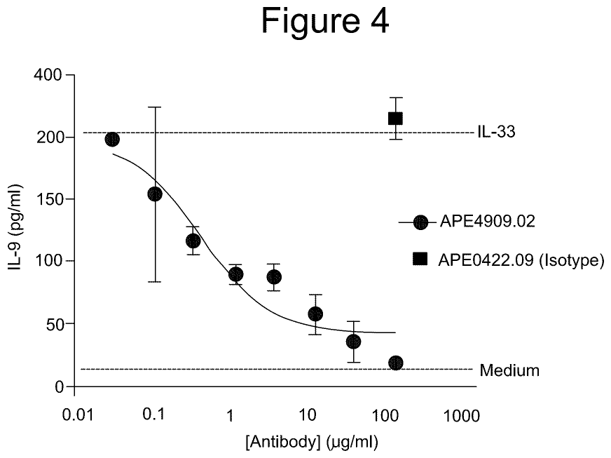

IL-5 and/or IL-9 levels in the cell supernatants were assessed by ELISA using a DUOSET™ ELISA kit (R&D Systems, Minneapolis, MN) following the manufacturer's suggested protocol. The APE4909 antibody inhibited IL-33 mediated release of IL-5 and IL-9 in primary human basophils, as shown in FIGS. 3 and 4.

The results of this example demonstrate that the inventive IL-33-binding agent can inhibit IL-33 activity.

### Example 3

This example demonstrates the affinity of an inventive IL-33 binding agent for IL-33.

The ability of the APE4909 antibody described in Example 2 to interact with IL-33 was analyzed biophysically using a KINEXA™ 3200 biosensor platform from Sapidyne Instruments (Boise, ID). Binding experiments for human and cynomolgus IL-33 (cynoIL-33) were conducted as described below and were run twice independently. Conditions for the first experiment are shown non-parenthetically, while conditions for the second experiment are provided parenthetically.

### APE4909/Human IL-33

Solid phase was prepared using azlactone-coated beads coated using a 50 g/mL solution of histidine-tagged human IL-33. Binding experiments were performed in 1×PBS pH 7.4, 0.1% BSA. The APE4909 antibody at 10 pM (or 20 pM) final concentration was incubated with IL-33 at 200 pM to 3.4 fM (or 400 pM to 6.7 fM) final concentrations for 3 (or 4) days at 4° C. 5 mL (or 10 mL) of each mixture was applied to the beads coated with IL-33 at a rate of 0.25 mL/min for 1200 seconds (or 2400 seconds). Free antibody was detected with an ALEXAFLUOR™ 647-(Life Technologies, Carlsbad, CA) labeled donkey anti-human antibody. All data fit using standard KINEXA™ software.

APE4909/Cyno IL-33 (cIL-33)

Experiments were performed as described above for human IL-33, except that the APE4909 antibody at 20 pM and 100 pM final concentration was incubated with cIL-33 at 3 nM to 315 fM final concentrations for 24 hours at 4° C. Each mixture was applied to the beads coated with cIL-33 at a rate of 0.25 mL/min for 500 seconds (for 20 pM) or 2120 seconds (for 100 pM). Free antibody was detected with an ALEXAFLUOR™ 647-(Life Technologies, Carlsbad, CA) labeled donkey anti-human antibody. The two curves were combined using N-curve analysis, and all data fit using KINEXA™ software. To verify, the experiment was repeated at 200 pM APE4909 antibody concentration using similarly prepared solid phase, buffers, and detection reagent. The APE4909 antibody was incubated with cIL-33 at 15 nM to 250 fM final concentrations for 24 hours at 4° C. and applied to the beads coated with cIL-33 at a rate of 0.25 mL/min for 180 seconds. This data fit using standard KINEXA™ software.

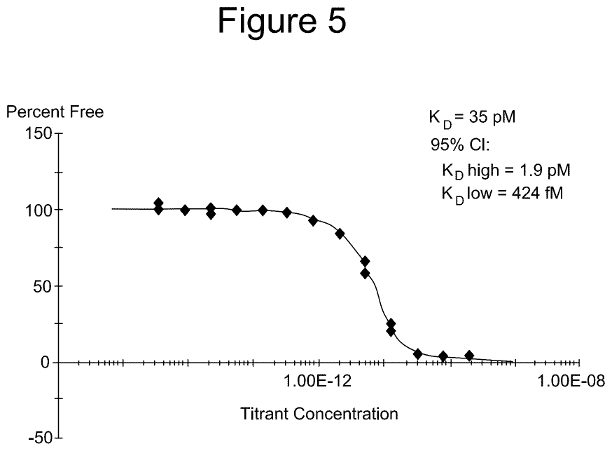

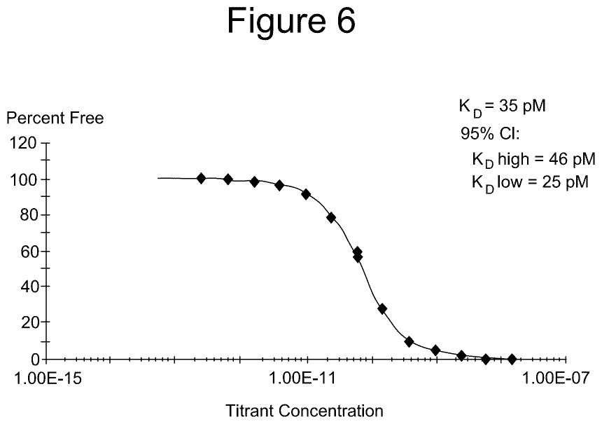

APE4909 affinities for human IL-33 and cynoIL-33 are shown FIG. 5 and FIG. 6, respectively. The results of this example demonstrate that the inventive IL-33-binding agent binds to both human IL-33 and non-human primate IL-33 with high affinity.

### Example 4

This example demonstrates that certain inventive IL-33-binding agents compete with the ST2 receptor for binding to human IL-33.

IL-33 binding was monitored using a BIACORE™ T200 system (GE Healthcare, Little Chalfont, Buckinghamshire, UK). Binding of IL-33 to various IL-33 antibodies disclosed herein or the human ST2 receptor was addressed by capturing an antibody and surveying the binding response of a fixed concentration of IL-33 in combination with increasing amounts of ST2. Anti-human IgG (Fc-specific, ˜10,000 RU) was immobilized on a BIACORE™ CM5 chip using EDC-activated amine coupling chemistry. The inventive IL-33 antibodies or human ST2 fused to a human IgG1 Fc region (2.0 μg/mL, 1 minute contact time at a flow rate of 10 L/min) were then captured at 25° C. (˜300 RU) onto this surface. Next, an analyte solution (pre-incubated for greater than 30 minutes) containing monomeric soluble human untagged IL-33 (1 nM) and untagged human ST2 (10, 3.3, 1.1 or 0.37 nM) was flowed over captured ligands for 2 minutes at a rate of 30 μL/min, and dissociation was monitored for an additional 2 minutes. The sensor chip surface was regenerated between each cycle using 3M MgCl₂ (60 seconds at 30 μL/min).

A second set of experiments was performed as described above, but with the following changes: (1) the sensor chip was immobilized with anti-mouse IgG (Fc-specific, ˜7500 RU); (2) human ST2 fused to murine IgG2aFc (1.0 μg/mL) was captured on the chip with a contact time of 4 minutes; and (3) the pre-incubated analyte solution contained monomeric soluble human untagged IL-33 (10 nM) and either an inventive IL-33 antibody or monomeric untagged human ST2 (100 nM, 25 nM, 6.3 nM or 1.6 nM). Analyte solution continued to incubate for approximately 30 minutes while the machine performed startup cycles. Capture and analyte binding was performed in HBS-EP+ running buffer (10 mM HEPES, pH 7.6, 150 mM NaCl, 3 mM EDTA, 0.05% Surfactant P-20; Teknova).

ST2 binding to the same epitope of IL-33 as an inventive antibody was associated with a loss of binding response, as the pre-incubation of ST2 with IL-33 would preclude access of the antibody to the epitope. ST2 binding to a different epitope of IL-33 permits IL-33 to bind the captured inventive antibody, which was observed as an increase in binding response since binding response is directly proportional to the mass of the analyte/complex. The results of these experiments are set forth in Table 1.

TABLE 1

| Inventive | | |
|---|---|---|
| Antibody | Response | Conclusion |
| APE00986 | no response in the absence of ST2 | does not bind IL-33 |
| APE02718 | response decreases as [ST2] | ST2 binds same/ |
| | increases | overlapping epitope |
| APE03833 | response decreases as [ST2] | ST2 binds same/ |
| | increases | overlapping epitope |
| APE04269 | response decreases as [ST2] | ST2 binds same/ |
| | increases | overlapping epitope |
| APE05492 | response increases as [ST2] | ST2 binds different |
| | increases | epitope |

The results of this example demonstrate that certain inventive IL-33-binding agents bind to an IL-33 epitope that is similar or identical to the epitope bound by the ST2 receptor.

### Example 5

This example demonstrates that an inventive IL-33-binding agent inhibits human IL-33-driven expansion of peripheral eosinophils.

IL-33 induces increased expression and release of IL-5 from CD4⁺ TH2 cell populations, innate lymphoid type-2 cells (ILC2 cells), and basophils. IL-5 is a cytokine that plays a key role in the differentiation, expansion, and survival of eosinophils, a population of cells that is known to mediate certain aspects of atopic disease indications, such as asthma and rhinitis. In a preliminary study, human IL-33 was injected intraperitoneally into wild-type Balb/c mice for six consecutive days at a dose of 5 μg per animal. Subsequent FACS analysis at the conclusion of this initial 6-day study indicated that human IL-33-treated mice had elevated numbers of cosinophils in their peripheral blood (as defined by high side-scatter analysis and CCR3 Siglec-F and CD16/CD32 expression) as compared to vehicle (PBS)-treated mice.

As a follow-up to the above study, wild-type Balb/c mice were again injected with 5 μg human IL-33 daily for 6-days total (days 1-6), and the anti-human IL-33 APE04909 antibody (described above) was administered on days-2 and +2 of the study at a dose of 10 mg/kg each day. Similar groups of mice treated with human IL-33 at a dose of 5 μg daily were administered either a control human IgG1 isotype mAb (designated APE00987) or human ST2-hFc fusion protein (designated APE027180), which represents a human IgG1 Fc-fusion dimeric version of the soluble IL-33 receptor (ST2). Both of these control proteins were also administered at 10 mg/kg doses on days-2 and +2 of the study only, as described for the APE04909 antibody.

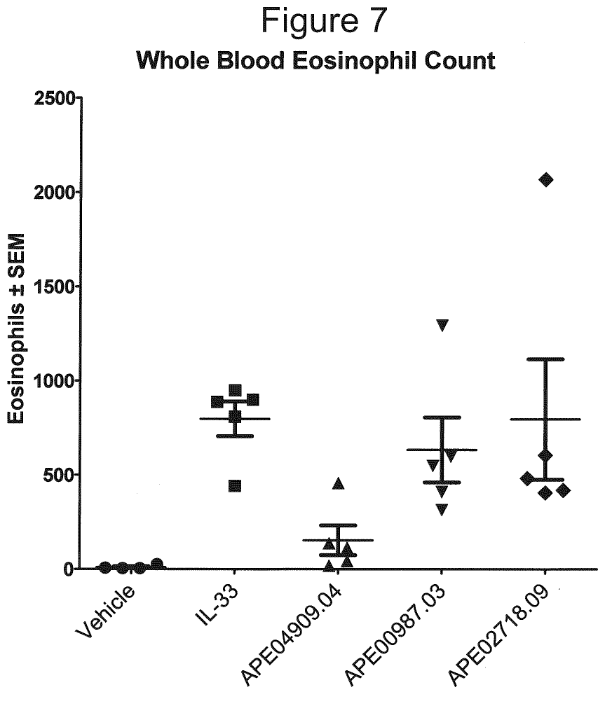

As shown in FIG. 7, the anti-IL-33 APE04909 antibody substantially inhibited human IL-33-driven cosinophil expansion in the peripheral blood compartment. In comparison, the human ST2-hFc protein failed to significantly reduce blood cosinophil numbers in human IL-33-treated mice and did not show a reduced level of eosinophil numbers above that detected in mice treated with a control IgG mAb (APE00987).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated immunoglobulin heavy chain polypeptide which comprises (a) an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, or (b) an amino acid sequence that is at least 90% identical to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217;
or
an isolated immunoglobulin light chain polypeptide which comprises (a) an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, or (b) an amino acid sequence that is at least 90% identical to any one of SEQ ID NO: 3, SEQ ID NO: 4, SEQ ID NOs: 51-66, SEQ ID NOs: 141-175, SEQ ID NOs: 189-205, and SEQ ID NOs: 218-231;
or
an isolated IL-33-binding agent which comprises (a) the immunoglobulin heavy chain polypeptide or at least one complementarity determining region (CDR) of the immunoglobulin heavy chain polypeptide, and/or (b) the immunoglobulin light chain polypeptide or at least one CDR of the immunoglobulin light chain polypeptide.

2. An isolated immunoglobulin heavy chain polypeptide, immunoglobulin light chain polypeptide, or IL-33 binding agent which binds to the same IL-33 epitope as the immunoglobulin heavy chain polypeptide, immunoglobulin light chain polypeptide, or IL-33 binding agent of claim 1.

3. (canceled)

4. (canceled)

5. An isolated or purified nucleic acid sequence encoding the isolated immunoglobulin heavy chain polypeptide, immunoglobulin light chain polypeptide, or IL-33 binding agent of claim 1.

6. (canceled)

7. A vector comprising the isolated or purified nucleic acid molecule of claim 5.

8. (canceled)

9. The isolated IL-33-binding agent of claim 1, which is an antibody, an antibody conjugate, or an antigen-binding fragment thereof.

10. The isolated IL-33-binding agent of claim 1, which is an antibody fragment selected from F(ab′)₂, Fab′, Fab, Fv, scFv, dsFv, dAb, and a single chain binding polypeptide.

11.-21. (canceled)

22. An isolated cell comprising the vector of claim 7.

23. A composition comprising (a) the isolated IL-33-binding agent of claim 1 or a nucleic acid encoding same and (b) a pharmaceutically acceptable carrier.

24. A method of treating a disorder in a mammal that is responsive to IL-33 inhibition, which method comprises administering an effective amount of the composition of claim 23 to a mammal having a disorder that is responsive to IL-33 inhibition, whereupon the disorder is treated in the mammal.

25. The method of claim 24, wherein the disorder is an inflammatory disorder.

26. The method of claim 25, wherein the inflammatory disorder is atopic dermatitis, allergic asthma, food allergy, or fibrosis.

27. The method of claim 26, wherein the food allergy is a peanut allergy.

28. The method of claim 24, wherein the disorder is an autoimmune disease.

29. The method of claim 28, wherein the autoimmune disease is Crohn's disease or rheumatoid arthritis.

30. The method of claim 24, wherein the disorder is cancer.

31. The method of claim 30, wherein the cancer is an epithelial cancer, chronic myelogenous leukemia (CML), breast cancer, or gastrointestinal cancer.

32. The method of claim 24, wherein the half-life of the IL-33-binding agent in the mammal is between 30 minutes and 45 days.

33. The method of claim 24, wherein the IL-33-binding agent binds to IL-33 with a K_{D} between about 1 femtomolar (fM) and about 100 micromolar (μM).
